(19) United States
(12) Patent Application Publication
(10) Pub. No.: US 2005/0282769 A1
(43) Pub. Date: Dec. 22, 2005

(54) Inhibitors of adenosine kinase for the treatment of optic nerve and retinal damage

(75) Inventor: Peter G. Klimko, Ft. Worth, TX (US)

Correspondence Address: Teresa J. Schultz;Alcon Research, Ltd., 6201 S. Freeway, Mail Code Q-148, Fort Worth, TX, 76134-2099, US

(73) Assignee: Alcon, Inc.

(21) Appl. No.: 11/183,447

(22) Filed: Jul. 18, 2005

Related U.S. Application Data
(63) Continuation of application No. 10/149,923, filed on Jun. 12, 2002, now abandoned.
(60) Provisional application No. 60/171,068, filed on Dec. 16, 1999.

(51) Int. Cl. A61K 31/7076; A61K 31/52; A61K 31/519

(52) U.S. Cl. 514/46; 514/263.2; 514/265.1

(57) ABSTRACT

Methods for preventing and treating damage to the optic nerve and/or retina with adenosine kinase inhibitors are disclosed.

This invention is directed to the treatment of optic nerve and retinal damage resulting from ischemia and hypoxia with compounds that inhibit the enzyme adenosine kinase (AK).

### BACKGROUND OF THE INVENTION

Retinal or optic nerve head damage, which can result in the loss of vision, can be caused by trauma and various pathological events including ischemia, hypoxia, or edema. There is increasing interest in pharmacological intervention using agents that treat instigators of the disease process, such as, nerve excitotoxicy or inappropriate oxygen consumption resulting from ischemia-reperfusion injury [for reviews, see Clark, Abbot F., Current trends in antiglaucoma therapy, Emerging Drugs, 4:333, 1999; David, Robert, Changing therapeutic paradigms in glaucoma management, Expert Opin. Invest. Drugs, 7:1063, 1998; Neuroprotection of the optic nerve in glaucoma, Acta Ophthalmol. Scand., 75:364, 1997].

The cytoprotective effects resulting from adenosine receptor activation, such as, vasodilation, neurotransmission inhibition, reduced oxygen consumption, and reduced inflammation are well known in the art [for reviews, see: Erion, M. D., Ann. Rep. Med. Chem., 28:295, 1993; DeNinno, M. P., Ann. Rep. Med. Chem., 33:111, 1998]. Treatment of various disease states characterized by reduced blood flow or inappropriately high neurotransmission rates (such as stroke, heart attack, and epilepsy) using systemic dosing of adenosine itself is generally not feasible because of its short half-life in the body and its side effect profile, the latter thought to be largely due to adenosine's lack of selectivity for binding to its endogenous receptors. A potential alternative strategy could be the use of compounds that are selective agonists at one of the adenosine receptor sub-types. In fact several such agents have been evaluated in animals and man for the treatment of damage resulting from stroke, brain trauma, and heart attack.

Another possible alternative is the use of compounds that inhibit the catabolism of adenosine. Cells in tissue suffering from ischemic stress increase their intracellular concentrations of adenosine by dephosphorylative catabolism of the important energy-storing molecule adenosine triphosphate (ATP). The adenosine so liberated diffuses out of the cell to bind to adenosine receptors and produce the ameliorative effects noted above [Jacobson, K. A., et. al., J. Med. Chem., 35:407, 1992]. Preventing adenosine catabolism should enhance this protective effect. Since cells in normal tissue usually have low intracellular concentrations of adenosine, this approach should have little effect in nontarget tissue [Tagetmeye, H., J. Mol. Cell. Cardiol., 17:1023, 1985]. One such method for increasing adenosine concentration is to inhibit AK-catalyzed phosphorylation of adenosine to adenosine monophosphate. As with the selective adenosine receptor agonists, several adenosine catabolizing-inhibiting compounds have been evaluated in animals and man for the treatment of damage resulting from stroke, brain trauma, and heart attack

Compounds which inhibit the uptake of adenosine have been disclosed for the treatment of retinal and optic neuropathy (Shade, U.S. Pat. No. 5,780,450), and a method for preventing retinal damage by administering a purine nucleoside analog has been disclosed (Gruber, U.S. Pat. No. 4,912,092). The effect of elevated adenosine concentration on tissue damage in animal models of retinal ischemia-reperfusion injury has been reviewed [Ghiardy, G. J., Gidday, J. M., and Roth, S., Vision Research, 39:2519, 1999]. Also, the use of nucleoside analogs as AK inhibitors, including the compounds of the present invention, to treat epilepsy, septic shock, ischemia-reperfusion injury, etc., has been disclosed (Firestein, G. S., et. al., WO 94/17803; Erion, M. D., et. al., U.S. Pat. No. 5,506,347; Browne, C. E., et. al., U.S. Pat. No. 5,864,033; Boyer, S. H., et. al,. U.S. Pat. No. 5,674,998; these are herein incorporated in their entirety by reference). Selected AK inhibitors have been shown to be effective anticonvulsants in a rat model of epilepsy [Erion, M. D., et. al., J. Pharmacol. Exp. Ther., 289:1669, 1999]. However, the compounds of the present invention are not known for the treatment of retinal and optic nerve damage resulting from ischemia or hypoxia.

### SUMMARY OF THE INVENTION

The present invention is directed to certain compounds that inhibit the enzyme AK for use in treating persons suffering from chronic or acute optic nerve and/or retinal damage. The present invention discloses compositions and methods for systemic, topical, and intraocular administration of at least one AK inhibitor in an amount effective to prevent or to treat retinal and/or optic nerve head tissue damage.

### DETAILED DESCRIPTION PREFERRED EMBODIMENTS

In accordance with the present invention and as used herein, the following terms are defined with the following meanings, unless explicitly stated otherwise.

As used herein, the terms “pharmaceutically acceptable salt” and “pharmaceutically acceptable ester” means any salt or ester, respectively, that would be suitable for therapeutic administration to a patient by any conventional means without significant deleterious health consequences; and “ophthalmically acceptable salt” and “ophthalmically acceptable ester” means any pharmaceutically acceptable salt or ester, respectively, that would be suitable for ophthalmic application, i.e. non-toxic and non-irritating. The compounds of Formula I are useful in both free base, free acid, and salt (protonated amine or carboxylate or phosphonate anion) form. In practice the use of a salt form amounts to use of the corresponding free acid or base form; all such forms are within the scope of the present invention.

The term “free hydroxy group” means an OH. The term “functionally modified hydroxy group” means an OH which has been functionalized to form: an ether, in which an alkyl, aryl, cycloalkyl, heterocycloalkyl, alkenyl, cycloalkenyl, heterocycloalkenyl, alkynyl, or heteroaryl group is substituted for the hydrogen; an ester, in which an acyl group is substituted for the hydrogen; a carbamate, in which an aminocarbonyl group is substituted for the hydrogen; or a carbonate, in which an aryloxy-, heteroaryloxy-, alkoxy-, cycloalkoxy-, heterocycloalkoxy-, alkenyloxy-, cycloalkenyloxy-, heterocycloalkenyloxy-, or alkynyloxy-carbonyl group is substituted for the hydrogen. Preferred moieties include OH, OPh, OCH₂C(O)CH₃, OCH₂C(O)C₂H₅, OCH₃, OCH₂CH₃, OC(O)CH₃, and OC(O)C₂H₅.

The term “free amino group” means an NH₂. The term “functionally modified amino group” means an NH₂ which has been functionalized to form: an aryloxy-, heteroaryloxy-, alkoxy-, cycloalkoxy-, heterocycloalkoxy-, alkenyl-, cycloalkenyl-, heterocycloalkenyl-, alkynyl-, or hydroxy-amino group, wherein the appropriate group is substituted for one of the hydrogens; an aryl-, heteroaryl-, alkyl-, cycloalkyl-, heterocycloalkyl-, alkenyl-, cycloalkenyl-, heterocycloalkenyl-, or alkynyl-amino group, wherein the appropriate group is substituted for one or both of the hydrogens; an amide, in which an acyl group is substituted for one of the hydrogens; a carbamate, in which an aryloxy-, heteroaryloxy-, alkoxy-, cycloalkoxy-, heterocycloalkoxy-, alkenyl-, cycloalkenyl-, heterocycloalkenyl-, or alkynyl-carbonyl group is substituted for one of the hydrogens; or a urea, in which an aminocarbonyl group is substituted for one of the hydrogens. Combinations of these substitution patterns, for example an NH₂ in which one of the hydrogens is replaced by an alkyl group and the other hydrogen is replaced by an alkoxycarbonyl group, also fall under the definition of a functionally modified amino group and are included within the scope of the present invention. Preferred moieties include NH₂, NHPh, NHCH₂Ph, NHCH₃, NHC₂H₅, N(CH₃)₂, NHC(O)CH₃, NHOH, and NH(OCH₃).

The term “free thiol group” means an SH. The term “functionally modified thiol group” means an SH which has been functionalized to form: a thioether, where an alkyl, aryl, cycloalkyl, heterocycloalkyl, alkenyl, cycloalkenyl, heterocycloalkenyl, alkynyl, or heteroaryl group is substituted for the hydrogen; or a thioester, in which an acyl group is substituted for the hydrogen. Preferred moieties include SH, SPh, SC(O)CH₃, SCH₃, SC₂H₅, SCH₂C(O)C₂H₅, and SCH₂C(O)CH₃.

The term “acyl” represents a group that is linked by a carbon atom that has a double bond to an oxygen atom and a single bond to another carbon atom.

The term “alkyl” includes straight or branched chain aliphatic hydrocarbon groups that are saturated and have 1 to 15 carbon atoms. The alkyl groups may be interrupted by one or more heteroatoms, such as oxygen, nitrogen, or sulfur, and may be substituted with other groups, such as halogen, hydroxyl, aryl, cycloalkyl, aryloxy, or alkoxy. Preferred straight or branched alkyl groups include methyl, ethyl, propyl, isopropyl, butyl and t-butyl.

The term “cycloalkyl” includes straight or branched chain, saturated or unsaturated aliphatic hydrocarbon groups which connect to form one or more rings, which can be fused or isolated. The rings may be substituted with other groups, such as halogen, hydroxyl, aryl, aryloxy, alkoxy, or lower alkyl. Preferred cycloalkyl groups include cyclopropyl, cyclobutyl, cyclopentyl and cyclohexyl.

The term “heterocycloalkyl” refers to cycloalkyl rings that contain at least one heteroatom such as O, S, or N in the ring, and can be fused or isolated. The rings may be substituted with other groups, such as halogen, hydroxyl, aryl, aryloxy, alkoxy, or lower alkyl. Preferred heterocycloalkyl groups include pyrrolidinyl, tetrahydrofuranyl, piperazinyl, and tetrahydropyranyl.

The term “alkenyl” includes straight or branched chain hydrocarbon groups having 1 to 15 carbon atoms with at least one carbon-carbon double bond, the chain being optionally interrupted by one or more heteroatoms. The chain hydrogens may be substituted with other groups, such as halogen. Preferred straight or branched alkenyl groups include allyl, 1-butenyl, 1-methyl-2-propenyl and 4-pentenyl.

The term “alkynyl” includes straight or branched chain hydrocarbon groups having 1 to 15 carbon atoms with at least one carbon-carbon triple bond, the chain being optionally interrupted by one or more heteroatoms. The chain hydrogens may be substituted with other groups, such as halogen. Preferred straight or branched alkynyl groups include ethynyl, propargyl, 1-butynyl, 1-methyl-2-propynyl and 4-pentynyl.

The term “cycloalkenyl” includes straight or branched chain, saturated or unsaturated aliphatic hydrocarbon groups which connect to form one or more non-aromatic rings containing a carbon-carbon double bond, which can be fused or isolated. The rings may be substituted with other groups, such as halogen, hydroxyl, alkoxy, or lower alkyl. Preferred cycloalkenyl groups include cyclopentenyl and cyclohexenyl.

The term “heterocycloalkenyl” refers to cycloalkenyl rings which contain one or more heteroatoms such as O, N, or S in the ring, and can be fused or isolated. The rings may be substituted with other groups, such as halogen, hydroxyl, aryl, aryloxy, alkoxy, or lower alkyl. Preferred heterocycloalkenyl groups include pyrrolidinyl, dihydropyranyl, and dihydrofuranyl.

The term “carbonyl group” represents a carbon atom double bonded to an oxygen atom, wherein the carbon atom has two free valencies.

The term “aminocarbonyl” represents a free or functionally modified amino group bonded from its nitrogen atom to the carbon atom of a carbonyl group, the carbonyl group itself being bonded to another atom through its carbon atom.

The term “lower alkyl” represents alkyl groups containing one to six carbons (C₁-C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, halogen, free or functionalized hydroxy, trihalomethyl, etc. Preferred aryl groups include phenyl, 3-(trifluoromethyl)phenyl, 3-chlorophenyl, and 4-fluorophenyl.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

The terms “aryloxy”, “heteroaryloxy”, “alkoxy”, “cycloalkoxy”, “heterocycloalkoxy”, “alkenyloxy”, “cycloalkenyloxy”, “heterocycloalkenyloxy”, and “alkynyloxy” represent an aryl, heteroaryl, alkyl, cycloalkyl, heterocycloalkyl, alkenyl, cycloalkenyl, heterocycloalkenyl, or alkynyl group attached through an oxygen linkage.

The terms “alkoxycarbonyl”, “aryloxycarbonyl”, “heteroaryloxycarbonyl”, “cycloalkoxycarbonyl”, “heterocycloalkoxycarbonyl”, “alkenyloxycarbonyl”, “cycloalkenyloxycarbonyl”, “heterocycloalkenyloxycarbonyl”, and “alkynyloxycarbonyl” represent an alkoxy, aryloxy, heteroaryloxy, cycloalkoxy, heterocycloalkoxy, alkenyloxy, cycloalkenyloxy, heterocycloalkenyloxy, or alkynyloxy group bonded from its oxygen atom to the carbon of a carbonyl group, the carbonyl group itself being bonded to another atom through its carbon atom.

The term “prodrug” as used herein refers to any compound that when administered to a biological system generates the “drug” substance either as a result of spontaneous chemical reaction(s) or by enzyme catalyzed or metabolic reaction(s). Reference is made to various prodrugs, such as alkyl, aralkyl, aryl, etc., esters, amides, carbonates, carbamates, and urethanes of carboxylic and phosphonic acids, and acylated or alkylated hydroxyl groups included herein. The groups illustrated are exemplary, not exhaustive and one skilled in the art could prepare other known varieties of prodrugs. Such prodrugs of the compounds of Formula I fall within the scope of the present invention.

The compounds which inhibit AK and are useful according to this invention are represented by Formula I: 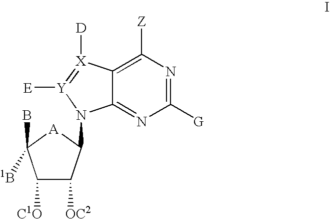
wherein:
A is oxygen, sulfur, or CH₂;
one of B, B¹ is H, and the other is alkenyl, alkynyl, or (CH₂)ₙB², where n is 1, 2, 3, or 4, and B² is H, alkyl, free or functionally modified hydroxy group, free or functionally modified amino group, free or functionally modified thiol group, N₃, CN, or halogen;
C¹O and C²O independently constitute a free or functionally modified hydroxy group, e.g., C¹ and C² independently are H, alkyl, acyl, or C¹ is a single bond to C² and C² is a carbonyl group;
X and Y are independently carbon or nitrogen, with the proviso that at least one of X and Y is carbon;
D is hydrogen, halogen, alkyl, aryl, aralkyl, alkenyl, alkynyl, free or functionalized hydroxy group, free or functionalized amino group, free or functionalized thiol group, CN, cyanoalkyl, CO₂H, alkoxycarbonyl, or aminocarbonyl when X is carbon, and is null when X is nitrogen;
E is hydrogen, halogen, alkyl, N₃, free or functionalized amino group, or free or functionalized thiol group when Y is carbon, and is null when Y is nitrogen;
Z is a free or functionalized amino group, hydrogen, halogen, a free or functionalized hydroxy group, a free or functionalized thiol group, aryl, CN, cyanoalkyl, or optionally substituted indolin-1-yl, indol-1-yl, pyrrolidin-1-yl, or piperaziny-1-yl;
G is hydrogen, halogen, free or functionalized amino group, free or functionalized thiol group,or free or functionalized hydroxy group;
and pharmaceutically acceptable salts and prodrugs thereof.

Compounds of the present invention can be synthesized according to the procedures detailed in WO 94/17803 and U.S. Pat. No. 5,506,347.

Included within the scope of the present invention are the individual enantiomers of the compounds of the present invention, as well as their racemic and non-racemic mixtures. The individual enantiomers can be enantioselectively synthesized from the appropriate enantiomerically pure or enriched starting material by means such as those described below. Alternatively, they may be enantioselectively synthesized from racemic/non-racemic or achiral starting materials. (Asymmetric Synthesis, J. D. Morrison and J. W. Scott, Eds., Academic Press Publishers: New York, 1983-1985, Volumes 1-5; Principles of Asymmetric Synthesis, R. E. Gawley and J. Aube, Eds., Elsevier Publishers: Amsterdam, 1996). They may also be isolated from racemic and non-racemic mixtures by a number of known methods, e.g. by purification of a sample by chiral HPLC (A Practical Guide to Chiral Separations by HPLC, G. Subramanian, Ed., VCH Publishers: New York, 1994; Chiral Separations by HPLC, A. M. Krstulovic, Ed., Ellis Horwood Ltd. Publishers, 1989), or by enantioselective hydrolysis of a carboxylic acid ester sample by an enzyme (Ohno, M., and Otsuka, M., Organic Reactions, Volume 37:1, 1989). Those skilled in the art will appreciate that racemic and non-racemic mixtures may be obtained by several means, including without limitation, nonenantioselective synthesis, partial resolution, or even mixing samples having different enantiomeric ratios. Departures may be made from such details within the scope of the accompanying claims without departing from the principles of the invention and without sacrificing its advantages. Also included within the scope of the present invention are the individual isomers substantially free of their respective enantiomers.

Preferred compounds of the present invention include those of formula I, wherein:
A is oxygen;
one of B, B¹ is H, and the other is CH₃, CH₂OH, or CH₂NH₂;
C¹O and C²O independently constitute a free or functionally modified hydroxy group, e.g., C¹ and C² independently are H, alkyl, acyl, or C¹ is a single bond to C² and C² is a carbonyl group;
Y is carbon or nitrogen;
X is carbon;
D is hydrogen, halogen, alkyl, aryl, or aralkyl;
E is hydrogen, halogen, or alkyl when Y is carbon and is null when Y is nitrogen;
Z is a free or functionalized amino group, hydrogen, halogen, aryl, or an optionally substituted indolin-1-yl, indol-1-yl, pyrrolidin-1-yl, or piperaziny-1-yl; and
G is hydrogen;
and pharmaceutically acceptable salts and prodrugs thereof.

Among the particularly preferred compounds of the present invention are the following compounds:

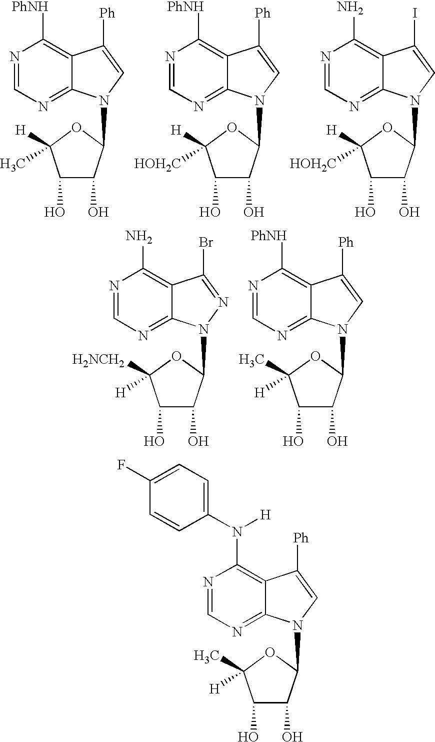

The compounds of the present invention significantly increase adenosine levels only in tissue undergoing hypoxic or ischemic stress, as these are the only sites which have significant adenosine concentrations due to net ATP breakdown via the ATP-AMP-adenosine pathway. Thus, side effects resulting from adenosine accumulation and receptor activation in non-target tissue should be greatly reduced compared to previously reported examples.

It is believed that compounds of Formula I are effective in preventing or treating damage to the retina and optic nerve, particularly damage resulting from ischemic or hypoxic stress, by elevating adenosine levels in the target tissue via inhibition of AK. The compounds are also useful for treating damage arising from the presence of cyto or neurotoxic entities, such as glutamate and other excitatory amino acids or peptides, excess intracellular calcium, and free radicals. In particular, the compounds can be useful in treating damage associated with branch and central vein/artery occlusion, anterior ischemic optic neuropathy, trauma, edema, angle-closure glaucoma, open-angle glaucoma, age related macular degeneration (ARMD), retinitis pigmentosa (RP), retinal detachments, damage associated with laser therapy, including photodynamic therapy (PDT), and surgical light induced iatrogenic retinopathy. The compounds may also be used as an adjunct to ophthalmic surgery, such as, by vitreal or subconjunctival injection following surgery. The compounds may also be used to treat acute conditions or prophylactically, especially prior to surgery or non-invasive procedures.

Using the above-described techniques, other AK inhibitor/s may become known, and are therefore, contemplated by the present invention and within the definition of AK inhibitors.

The AK inhibitor/s may be contained in various types of pharmaceutical compositions, in accordance with formulation techniques known to those skilled in the art. For example, the compounds may be included in tablets, capsules, solutions, suspensions, and other dosage forms adapted for oral administration; solutions and suspensions adapted for parenteral use; and solutions and suspensions adapted for topical ophthalmic, depot, or intra-ocular injection. Solutions, suspensions, and other dosage forms adapted for depot, oral, intra-ocular injection, and topical ophthalmic administration, such as eye drops or tissue irrigating solutions, are particularly preferred for the prevention or treatment of acute or chronic retinal or optic nerve head damage. Compositions can also be delivered topical ophthalmically according to the teachings in WO 96/05840, which is herein incorporated by reference.

The present invention is particularly directed to the provision of compositions adapted for treatment of retinal and optic nerve head tissues. The ophthalmic compositions of the present invention will include one or more AK inhibitor/s and a pharmaceutically acceptable vehicle. Various types of vehicles may be used. The vehicles will generally be aqueous in nature. Aqueous solutions are generally preferred, based on ease of formulation, as well as a patient's ability to easily administer such compositions by means of instilling one to two drops of the solutions in the affected eyes. However, the AK inhibitor/s of the present invention may also be readily incorporated into other types of compositions, such as suspensions, viscous or semi-viscous gels, or other types of solid or semi-solid compositions. Suspensions may be preferred for AK inhibitor/s that are relatively insoluble in water. The ophthalmic compositions of the present invention may also include various other ingredients, such as buffers, preservatives, co-solvents, and viscosity building agents.

An appropriate buffer system (e.g., sodium phosphate, sodium acetate or sodium borate) may be added to prevent pH drift under storage conditions.

Ophthalmic products are typically packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquatemium-1, or other agents known to those skilled in the art. Such preservatives are typically employed at a level of from 0.001 to 1.0% weight/volume (“% w/v”).

When the AK inhibitor/s of the present invention are administered during intraocular surgical procedures, such as through retrobulbar or periocular injection and intraocular perfusion or injection, the use of balanced salt irrigating solutions as vehicles are most preferred. BSS® Sterile Irrigating Solution and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022 (Garabedian, et al.), the entire contents of which are hereby incorporated in the present specification by reference. Retrobulbar and periocular injections are known to those skilled in the art and are described in numerous publications including, for example, Ophthalmic Surgery: Principles of Practice, Ed., G. L. Spaeth, W. B. Sanders Co., Philadelphia, Pa., U.S.A., pg. 85-87, 1990.

The route of administration (e.g., topical, ocular injection, parenteral, or oral) and the dosage regimen will be determined by skilled clinicians, based on factors such as the exact nature of the condition being treated, the severity of the condition, and the age and general physical condition of the patient.

In general, the doses used for the above described purposes will vary, but will be in an effective amount to prevent, reduce or ameliorate retinal or optic nerve head tissue damage resulting from any of the above listed conditions. As used herein, the term “pharmaceutically effective amount” refers to an amount of one or more AK inhibitor/s which will prevent, reduce, or ameliorate chronic or acute retinal or optic nerve head damage resulting from ischemic or hypoxic conditions in a human patient. The doses used for any of the above-described purposes will generally be from about 0.01 to about 100 milligrams per kilogram of body weight (mg/kg), administered one to four times per day. When the compositions are dosed topically, they will generally be in a concentration range of from 0.001 to about 5% w/v, with 1-2 drops administered 1-4 times per day.

As used herein, the term “pharmaceutically acceptable carrier” refers to any formulation that is safe, and provides the appropriate delivery for the desired route of administration of an effective amount of at least one compound of the present invention.

The following Examples 1 and 2 are formulations useful for intraocular, periocular, or retrobulbar injection or perfusion.

### EXAMPLE 1

| Component | % w/v |
|---|---|
| AK inhibitor | 0.1 |
| Dibasic sodium phosphate | 0.2 |
| HPMC | 0.5 |
| Polysorbate 80 | 0.05 |
| Benzalkonium chloride | 0.01 |
| Sodium chloride | 0.75 |
| Edetate disodium | 0.01 |
| NaOH/HCI | q.s. to pH 7.4 |
| Purified water | q.s. to 100% |

### EXAMPLE 2

| Component | % w/v |
|---|---|
| AK inhibitor | 0.1 |
| Cremophor EL | 10 |
| Tromethamine | 0.12 |
| Boric acid | 0.3 |
| Mannitol | 4.6 |
| Edetate disodium | 0.1 |
| Benzalkonium chloride | 0.1 |
| NaOH/HCI | q.s. to pH 7.4 |
| Purified water | q.s. to 100% |

### EXAMPLE 3

An AK inhibitor/s of the present invention can be formulated in an ocular irrigating solution used during ophthalmic surgery to treat retinal or optic nerve head damage resulting from trauma due to injury or prevent damages resulting from the invasive nature of the surgery. The concentration of the AK inhibitor/s in the irrigating solution will range from 0.001 to 5% w/v.

### EXAMPLE 4

The following tablet formulation can be made pursuant to U.S. Pat. No. 5,049,586, incorporated herein by reference.

| Component | % w/v |
|---|---|
| AK inhibitor | 60 |
| Magnesium oxide | 20 |
| Corn starch | 15 |
| Polyvinylpyrrolidone | 3 |
| Sodium carboxymethylcellulose | 1 |
| Magnesium stearate | 0.8 |

1: A method for treating damage to the optic nerve head or retina resulting from ischemia or hypoxia, said method comprising administering a composition comprising a compound which inhibits adenosine kinase, wherein the compound has the following formula: 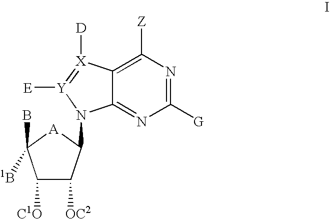
wherein:
A is oxygen, sulfur, or CH₂;
one of B, B¹ is H, and the other is alkenyl, alkynyl, or (CH₂)ₙB², where n is 1, 2, 3, or 4, and B² is H, alkyl, free or functionally modified hydroxy group, free or functionally modified amino group, free or functionally modified thiol group, N₃, CN, or halogen;
C¹O and C²O independently constitute a free or functionally modified hydroxy group, e.g. C¹ and C² independently are H, alkyl, acyl, or C¹ is a single bond to C² and C² is a carbonyl group;
X and Y are independently carbon or nitrogen, with the proviso that at least one of X and Y is carbon;
D is hydrogen, halogen, alkyl, aryl, aralkyl, alkenyl, alkynyl, free or functionalized hydroxy group, free or functionalized amino group, free or functionalized thiol group, CN, cyanoalkyl, CO₂H, alkoxycarbonyl, or aminocarbonyl when X is carbon, and is null when X is nitrogen;
E is hydrogen, halogen, alkyl, N₃, free or functionalized amino group, or free or functionalized thiol group when Y is carbon, and is null when Y is nitrogen;
Z is a free or functionalized amino group, hydrogen, halogen, a free or functionalized hydroxy group, a free or functionalized thiol group, aryl, CN, cyanoalkyl, or optionally substituted indolin-1-yl, indol-1-yl, pyrrolidin-1-yl, or piperaziny-1-yl;
G is hydrogen, halogen, a free or functionalized amino group, a free or functionalized thiol group, or a free or functionalized hydroxy group;
and pharmaceutically acceptable salts and prodrugs thereof, in a pharmaceutically acceptable carrier, and wherein the concentration of the compound in the composition is from about 0.001% to about 5% w/v.

2. (canceled)

3. (canceled)

4: The method of claim 1 wherein the damage is associated with a condition selected from the group consisting of branch and central vein/artery occlusion, angle-closure glaucoma, open-angle glaucoma, anterior ischemic optic neuropathy, ARMD, RP, retinal detachments, laser therapy, and surgical light induced iatrogenic retinopathy.

5: The method of claim 1, wherein for the compound:
A is oxygen;
one of B, B¹ is H, and the other is CH₃, CH₂OH, or CH₂NH₂;
C¹O and C²O independently constitute a free or functionally modified hydroxy group, e.g. C¹ and C² independently are H, alkyl, acyl, or C¹ is a single bond to C² and C² is a carbonyl group;
Y is carbon or nitrogen;
X is carbon;
D is hydrogen, halogen, alkyl, aryl, or aralkyl;
E is hydrogen, halogen, or alkyl when Y is carbon and is null when Y is nitrogen;
Z is a free or functionalized amino group, hydrogen, halogen, aryl, or an optionally substituted indolin-1-yl, indol-1-yl, pyrrolidin-1-yl, or piperaziny-1-yl; and
G is hydrogen.

6: The method of claim 5, wherein the compound is selected from the group consisting of:

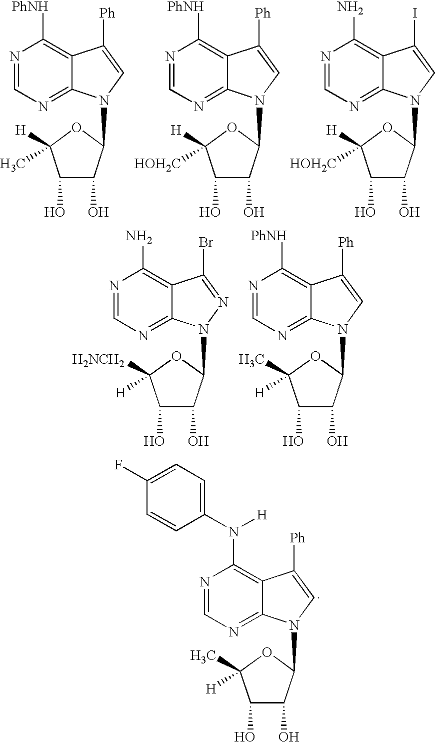

7. (canceled)

8. (canceled)

9-12. (canceled)
